SEQUENCE LISTING (4 sequences)

SEQ ID NO: 1 - DNA, 70 bp, Artificial Sequence
  synthetic construct
cacttctagaatgtacccatacgatgttccagattacgctgaacaagccccagaagaccaagggccacag

SEQ ID NO: 2 - DNA, 32 bp, artificial sequence
  synthetic construct
cactggatcctaggatctactggctccatttc

SEQ ID NO: 3 - DNA, 32 bp, artificial sequence; identical to SEQ ID NO: 2
  synthetic contruct

SEQ ID NO: 4 - DNA, 31 bp, artificial sequence
  synthetic construct
ataaacagcagttgtcgcagaattcttatta

(19) United States
(12) Patent Application Publication
(10) Pub. No.: US 2013/0109687 A1
(43) Pub. Date: May 2, 2013

(54) METHODS OF TREATING HIV INFECTION: INHIBITION OF DNA DEPENDENT PROTEIN KINASE

(71) Applicants: Gary J. Nabel, Washington, DC (US); Arik Cooper, Washington, DC (US)

(72) Inventors: Gary J. Nabel, Washington, DC (US); Arik Cooper, Washington, DC (US)

(21) Appl. No.: 13/643,819

(22) PCT Filed: May 2, 2011
(86) PCT No.: PCT/US11/34774

§ 371 Date: Nov. 26, 2012

(30) Foreign Application Priority Data
Apr. 30, 2010 (US) 61329775

(51) Int. Cl. C07D 409/10; C07D 311/92

(52) U.S. Cl. CPC C07D 409/10; C07D 311/92
USPC 514/233.5; 514/232.8

(57) ABSTRACT

Methods of treating HIV-1 infection/AIDS in a patient infected with an HIV-1 virus comprising providing a DNA-PK inhibitor to the patient are provided herein. In one embodiment the DNA-PK inhibitor is compound of the Formula I

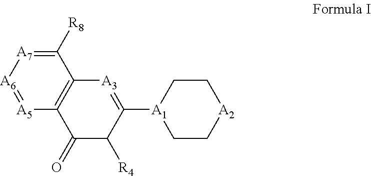

or a pharmaceutically acceptable salt thereof. The variables in Formula I, e.g. A₁, A₂, A₃, R₄, A₅, A₆, A₇, and R₈, are described herein.

### CROSS REFERENCE TO RELATED APPLICATION

This application claims priority from U.S. Provisional Patent application No. 61/329,775, filed Apr. 30, 2010, which is hereby incorporated by reference in its entirety.

### STATEMENT OF GOVERNMENT SUPPORT

This invention was made in part with government support from the National Institutes of Health. The government has certain rights in this invention.

### FIELD OF THE DISCLOSURE

The inventors have discovered that a cellular kinase, DNA Dependent Protein Kinase (DNAPK) mediates killing of CD4 T cells by HIV-1. NU7026, a chemical inhibitor of DNAPK is shown to cause a substantial reduction in cell death following HIV infection in vitro. Methods of treating HIV infection/AIDS by providing a DNAPK inhibitor to a patient infected with an HIV-1 virus are provided herein. The DNAPK inhibitor may be NU7026, NU7441, or close structural analogues of these compounds as described herein.

### BACKGROUND

Human immunodeficiency virus-1 (HIV-1) has infected more than 60 million people and caused nearly 30 million deaths, ultimately the consequence of catalytic replication of the virus in CD4⁺ cells¹⁻³.

A hallmark of HIV-1 infection in the progressive loss of CD4⁺ cells that leads ultimately to immunodeficiency. Despite the profound consequences of immunodeficiency, the mechanism by which HIV-1 induces CD4⁺ T-cell death remains unknown. While HAART restores T cell counts in infected patients, such therapy does not eliminate the virus from persistent reservoirs and potential emergence of HAART resistant HIV-1 strains necessitates development of new drug with novel mechanisms of actions.

### SUMMARY

Methods of treating HIV-1 infection/AIDS in a patient infected with an HIV-1 virus comprising providing a therapeutically effective amount of DNA-PK inhibitor to the patient are provided herein.

In one embodiment the DNA-PK inhibitor is a compound of the Formula I

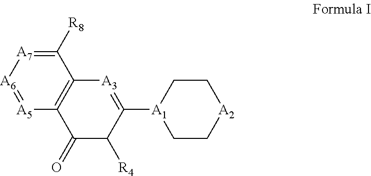

or a pharmaceutically acceptable salt thereof. Within Formula I the variables, i.e., A₁, A₂, A₃, R₄, A₅, A₆, A₇, and R₈ carry the following definitions.

A₁ is N or CH; A₂ is NH, O, S, or CH; and A₃ is NH, O, S, or CH.

R₄ is hydrogen, halogen, hydroxyl, cyano, amino, thiol, phenyl, C₁-C₄alkyl, C₁-C₄alkoxy, mono- or di-C₁-C₄alkylamino, C₁-C₂haloalkyl, or C₁-C₂haloalkoxy.

A₅ is N or CR₅; A₆ is N or CR₆; and A₇ is N or CR₇; wherein not more than 2 of A₅, A₆, and A₇ are N.

R₅ and R₆ are independently chosen at each occurrence from hydrogen, halogen, hydroxyl, cyano, amino, thiol, C₁-C₄alkyl, C₁-C₄alkoxy, mono- or di-C₁-C₄alkylamino, C₁-C₂haloalkyl, C₁-C₂haloalkoxy, C₃-C₇cyclolkyl, heterocycloalkyl having 5 to 7 ring atoms with 1 or 2 ring atoms being, N, S, or O and remaining ring members being carbon.

R₇ carries the same definition as R₅ and R₆ or may be joined with R₈ to form a ring; and R₈ is an optionally substituted heterocyclic or carbocyclic ring system having one ring or two or three fused rings, each ring containing from 0 to 3 heteroatoms independently chosen from N, O, and S; or R₇ and R₈ are joined to form an optionally substituted phenyl or pyridyl ring.

### BRIEF DESCRIPTION OF THE DRAWINGS

FIG. 1. Dissociation of VPR-induced Bax- and caspase-dependent apoptosis and G2/M cell cycle arrest in 293T cells, and protection against apoptosis by G1/S growth arrest. FIG. 1a, Percentage of apoptotic cells with DNA content less than diploid levels (left) and cell cycle distribution (right) in cells transduced with a lentiviral vector encoding either a point mutant, Q65R, or wild type VPR as determined by flow cytometric analysis of DNA content using propidium iodide (PI) staining. FIG. 1b, G1/S growth arrest protects against apoptotic effects of VPR. The percentage of apoptotic cells in the absence or presence of p27 overexpression are shown (left), and the effect on cell cycle progression is indicated (right). FIG. 1c, Percentage of apoptotic cells (left) and cell cycle distribution (right) in cells transduced with a lentiviral vector encoding wild type VPR and treated with DMSO or 100 μM Z-VAD-fmk, as determined in (a). FIG. 1d, Percentage of apoptotic cells (left) and cell cycle distribution (right) in cells transfected with non-targeting, control siRNA or siRNA targeting Bax and transduced with a lentiviral vector encoding wild type VPR.

FIG. 2. VPR binds and activates DNA-PK. FIG. 2a, Western blot analysis of DNA-PKcs-associated Ku70, Ku80, and VPR from cells transfected with an empty vector or a VPR-encoding vector (lanes 1-6, as indicated). FIG. 2b, Western blot analysis of DNA-PKcs immunoprecipitates showing input (lanes 1,2) and association of VPR with DNA-PKcs following control treatment or immunodepletion of VPRBP (lanes 3 vs. 4 respectively) FIG. 2c, Western blot analysis of DNA-PKcs immunoprecipitates showing association of wild type, but not Q65R mutant VPR with DNA-PKcs. FIG. 2d, VPR induction of DNA-PKcs autophosphorylation (left, lanes 1,2) and Western blot analysis of γH2AX from cells transfected with a VPR-encoding plasmid and treated with the indicated concentrations of DNA-PK inhibitor NU7026 (right, lanes 3-7). Western blot analysis of autophosphorylated DNA-PKcs was performed using anti-phosphoserine 2056 DNA-PKcs from cells transfected with an empty vector or a VPR-encoding plasmid. Cell lysates were subjected to Western blotting using the indicated antibodies.

FIG. 3. Apoptosis induced by VPR requires DNA-PK: effect of DNA-PK catalytic subunit inhibitor and siRNA knockdown. FIG. 3a, Quantitation of apoptotic cell death (left) and cell cycle distribution (right) by DNA content analysis using propidium iodide staining in 293T cells mock-treated or transduced with a lentiviral vector encoding VPR and treated with DMSO or DNA-PK inhibitor NU7026 (10 μM). Cells were harvested 72 h after transduction and background apoptosis from the respective controls was subtracted for each condition. FIG. 3b, Quantitation of apoptotic cell death (middle) and cell cycle distribution (right) by DNA content analysis using propidium iodide staining in 293T cells transfected with a non-targeting siRNA (control) or siRNA directed against DNA-PKcs and either mock infected or transduced with a lentiviral vector encoding VPR. Cells were harvested 48 hrs after transduction. Knockdown of DNA-PKcs protein was confirmed by Western blotting (left). Background apoptosis from the respective controls was subtracted for each condition.

FIG. 4. Cytopathicity induced by HIV-1 replication in primary human CD4⁺ T cells is mediated by VPR and dependent on DNA-PK:effect of DNA-PK inhibitor and siRNA knockdown. FIG. 4a, Viability analysis of primary CD4⁺ T cells infected with wild type or ΔVPR HIV-1_{89.6}. 12 days after infection the cells were harvested, stained with anti-p24, Annexin V and Vivid, and analyzed by flow cytometry. Early apoptotic cells were characterized by positive staining for Annexin V and lack of reactivity with Vivid. Late apoptotic cells are characterized by double positivity. The percentage of apoptotic cells is circled in red. The experiment is representative of three independent experiments using three different donors. FIG. 4b, Viability analysis of primary CD4⁺ T cells infected with wild type HIV-1_{89.6} in the presence of DMSO or DNA-PK inhibitor NU7026. 8 days after infection, the cells were harvested, stained with anti-p24, Annexin V and Vivid, and analyzed by flow cytometry. The experiment is representative of two independent experiments using three different donors. FIG. 4c, Primary CD4s were nucleofected with either non-targeting siRNA (control) or siRNA directed against DNA-PK and infected with wild type or A VPR HIV-1_{89.6}. 10 days after infection, the cells were harvested, stained with anti-p24, Annexin V and Vivid, and analyzed by flow cytometry.

FIG. 5. Infected cells were identified by p24 staining, and viability was monitored using combined staining with Annexin V and the amine reactive viability dye ViVid. FIG. 5a, Replication of both viruses was comparable as revealed by similar levels of p24 staining. FIG. 5b, The frequency of infection, determined by p24 positivity using flow cytometry, remained unchanged by treatment with DNA-PK inhibitor excluding the possibility that decreased viral infectivity accounted for the diminished death. FIG. 5c, DNA-PKcs knockdown did not affect the infectivity of either virus.

### DETAILED DESCRIPTION

### Terminology

Prior to setting forth the invention in detail, it may be helpful to provide definitions of certain terms to be used herein. Compounds are generally described using standard nomenclature.

The present invention is intended to include all isotopes of atoms occurring in the present compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C.

Certain compounds are described herein using a general formula that includes variables, e.g., A₁, R₂, R₃, R₅, A₆, R₇, A₈, and R₉. Unless otherwise specified, each variable within such a formula is defined independently of other variables. Thus, if a group is said to be substituted, e.g., with 0-2 R*, then said group may be substituted with up to two R* groups and R* at each occurrence is selected independently from the definition of R*. Also, combinations of substituents and/or variables are permissible only if such combinations result in stable compounds. When a group is substituted by an “oxo” substituent, a carbonyl bond replaces two hydrogen atoms on a carbon. An “oxo” substituent on an aromatic group or heteroaromatic group destroys the aromatic character of that group, e.g., a pyridyl substituted with oxo is a pyridone.

The term “substituted” as used herein means that any one or more hydrogen atoms bound to the designated atom or group is replaced with a selection from the indicated group, provided that the designated atom's normal valence is not exceeded. When a substituent is oxo (i.e., ═O), then 2 hydrogen atoms on the substituted atom are replaced with a double-bonded oxygen. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent. Unless otherwise specified, substituents are named into the core structure. For example, it is to be understood that when (cycloalkyl)alkyl is listed as a possible substituent the point of attachment of this substituent to the core structure is in the alkyl portion.

“Alkyl” includes both branched and straight-chain saturated aliphatic hydrocarbon groups having the specified number of carbon atoms. Thus, the term C₁-C₆alkyl as used herein includes alkyl groups having from 1 to about 6 carbon atoms. When C₀-Cₙ alkyl is used herein in conjunction with another group, for example, (aryl)C₀-C₄ alkyl, the indicated group, in this case aryl, is either directly bound by a single covalent bond (C₀), or attached by an alkyl chain having the specified number of carbon atoms, in this case from 1 to about 4 carbon atoms. Examples of alkyl include, but are not limited to, methyl, ethyl, n-propyl, isopropyl, n-butyl, t-butyl, n-pentyl, and sec-pentyl. C₁-C₆alkyl includes alkyl groups have 1, 2, 3, 4, 5, or 6 carbon atoms.

“Alkoxy” is an alkyl group as defined above with the indicated number of carbon atoms attached through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

“Mono- and/or di-alkylamino” are secondary or tertiary alkyl amino groups, wherein the alkyl groups are as defined above and have the indicated number of carbon atoms. The point of attachment of the alkylamino group is on the nitrogen. Examples of mono- and di-alkylamino groups include ethylamino, dimethylamino, and methyl-propyl-amino.

A “carbocyclic group” is a monocyclic, bicyclic or tricyclic saturated, partially unsaturated, or aromatic ring system in which all ring atoms are carbon. Usually each ring of the carbocyclic group contains from 4-6 ring atoms and a bicyclic carbocyclic group contains from 7 to 10 ring atoms but some other number of ring atoms may be specified. Unless otherwise indicated, the carbocyclic group may be attached to the group it substitutes at any carbon atom that results in a stable structure. When indicated the carbocyclic rings described herein may be substituted at any carbon atom if the resulting compound is stable.

“Cycloalkyl” is a saturated hydrocarbon ring group, having the specified number of carbon atoms, usually from 3 to about 8 ring carbon atoms, or from 3 to about 7 carbon atoms. Examples of cycloalkyl groups include cyclopropyl, cyclobutyl, cyclopentyl, or cyclohexyl as well as bridged or caged saturated ring groups such as norborane or adamantane.

“Haloalkyl” includes both branched and straight-chain saturated aliphatic hydrocarbon groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl. “Haloalkoxy” indicates a haloalkyl group as defined above attached through an oxygen bridge.

“Halo” or “halogen” as used herein refers to fluoro, chloro, bromo, or iodo.

“Heteroaryl” indicates a stable 5- to 7-membered monocyclic or 7- to 10-membered bicyclic heterocyclic ring which contains at least 1 aromatic ring that contains from 1 to 4, or preferably from 1 to 3, heteroatoms chosen from N, O, and S, with remaining ring atoms being carbon. When the total number of S and O atoms in the heteroaryl group exceeds 1, these heteroatoms are not adjacent to one another. It is preferred that the total number of S and O atoms in the heteroaryl group is not more than 2. It is particularly preferred that the total number of S and O atoms in the heteroaryl group is not more than 1. A nitrogen atom in a heteroaryl group may optionally be quaternized. When indicated, such heteroaryl groups may be further substituted with carbon or non-carbon atoms or groups. Such substitution may include fusion to a 5 to 7-membered saturated cyclic group that optionally contains 1 or 2 heteroatoms independently chosen from N, O, and S, to form, for example, a [1,3]dioxolo[4,5-c]pyridyl group. Examples of heteroaryl groups include, but are not limited to, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, benz[b]thiophenyl, isoquinolinyl, quinazolinyl, quinoxalinyl, thienyl, isoindolyl, and 5,6,7,8-tetrahydroisoquinoline.

“Heterocycloalkyl” indicates a saturated cyclic group containing from 1 to about 3 heteroatoms chosen from N, O, and S, with remaining ring atoms being carbon. Heterocycloalkyl groups have from 3 to about 8 ring atoms, and more typically have from 5 to 7 ring atoms. Examples of heterocycloalkyl groups include morpholinyl, piperazinyl, piperidinyl, and pyrrolidinyl groups.

The term “heterocyclic group” indicates a monocyclic saturated, partially unsaturated, or aromatic ring containing from 1 to about 4 heteroatoms chosen from N, O, and S, with remaining ring atoms being carbon, or a bicyclic saturated, partially unsaturated, or aromatic heterocylic ring system containing at least 1 heteroatom in the two ring system chosen from N, O, and S and containing up to about 4 heteroatoms independently chosen from N, O, and S in each ring of the two ring system. Usually each ring of the heterocyclic group contains from 4-6 ring atoms but some other number of ring atoms may be specified. Unless otherwise indicated, the heterocyclic ring may be attached to its pendant group at any heteroatom or carbon atom that results in a stable structure. When indicated the heterocyclic rings described herein may be substituted on carbon or on a nitrogen atom if the resulting compound is stable. It is preferred that the total number of heteroatoms in a heterocyclic groups is not more than 4 and that the total number of S and O atoms in a heterocyclic group is not more than 2, more preferably not more than 1. Examples of heterocyclic groups include, dibenzothiophenyl, dibenzofuranyl, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, benz[b]thiophenyl, isoquinolinyl, quinazolinyl, quinoxalinyl, thienyl, isoindolyl, dihydroisoindolyl, 5,6,7,8-tetrahydroisoquinoline, pyridinyl, pyrimidinyl, furanyl, thienyl, pyrrolyl, pyrazolyl, pyrrolidinyl, morpholinyl, piperazinyl, piperidinyl, and pyrrolidinyl.

“Salts” of the compounds of the compounds disclosed herein include inorganic and organic acid and base addition salts. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Salts of the present compounds further include solvates of the compounds and of the compound salts.

“Pharmaceutically acceptable salts” includes derivatives of the disclosed compounds wherein the parent compound is modified by making non-toxic acid or base salts thereof, and further refers to pharmaceutically acceptable hydrates solvates of such compounds and such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxylmaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in Remington's Pharmaceutical Sciences, 17th ed., Mack Publishing Company, Easton, Pa., p. 1418 (1985).

A “therapeutically effective amount” of a compound of Formula I, or a related formula, is an amount effective, when administered to a patient, to provide a therapeutic benefit such as an amelioration of symptoms, e.g., an amount effective to decrease the symptoms of a viral infection. In certain circumstances a patient suffering from a viral infection may not present symptoms of being infected. Thus a therapeutically effective amount of a compound is also an amount sufficient significantly reduce the detectable level of virus or viral antibodies against the microorganism in the patient's blood, serum, other bodily fluids, or tissues.

### DESCRIPTION

The HIV-1 accessory protein VPR mediates cell death in infected CD4 lymphocytes through activation of DNA-Dependent Protein Kinase (DNA-PK), a cellular kinase normally involved in non-homologous end-joining of double-stranded DNA breaks⁴. The inventors hereof have discovered that replication of wild type, but not VPR-deficient, HIV-1 promotes the death of primary CD4⁺ lymphocytes. Expression of VPR alone similarly increases cell death. VPR interacts with the catalytic subunit of DNA-PK (DNA-PKcs) independently of VPR binding protein (VprBP). This interaction stimulates DNA-PK activity measured by phosphorylation of a known DNA-PK substrate, histone H2AX⁵. Inhibition or knockdown of DNA-PK strikingly reduces VPR-induced cell death in HIV-1 infected human T lymphocytes. Activation of DNA-PK by VPR plays a central role in CD4⁺ T-cell depletion, suggesting that interventions directed towards this cellular kinase may improve T-cell survival and immune function in HIV-1 infected individuals.

A hallmark of HIV-1 infection is the progressive loss of CD4⁺ T cells that leads ultimately to immunodeficiency. Factors including chronic immune hyper-activation, disruption of CD4⁺ T-cell homeostasis, direct killing of infected cells as well as indirect (bystander) death of uninfected cells may potentially contribute to the CD4⁺ T-cell deficit^{6,7}. While no single factor is responsible for CD4⁺ cell destruction, direct cytotoxicity exerted by the replicating virus in infected cells contributes prominently to T-cell depletion^{8,9}, and studies in macaques using simian lentiviruses have shown that the majority of CD4⁺ T cells are infected and rapidly eliminated at the peak of viremia^{2,3}. However, the mechanism of virus-induced cytotoxicity remains unknown. Among the HIV-1 gene products, VPR exerts effects on transcription and the stress response during replication in CD4 cells, promoting G2/M cell cycle arrest¹⁰. VPR also causes apoptosis in a variety of cell types¹⁰, yet direct evidence demonstrating the relevance of this phenotype to depletion of primary CD4 cells following HIV-1 infection is lacking. Applicants have determined that VPR is responsible for the death of CD4 cells caused by viral infection.

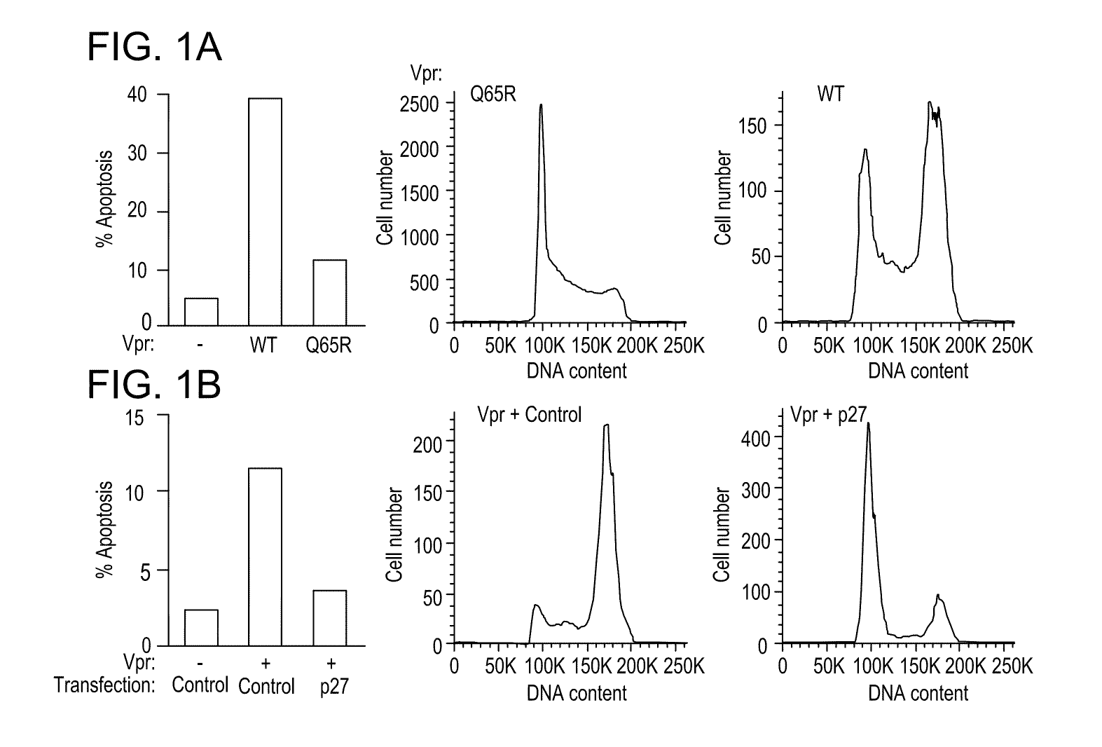

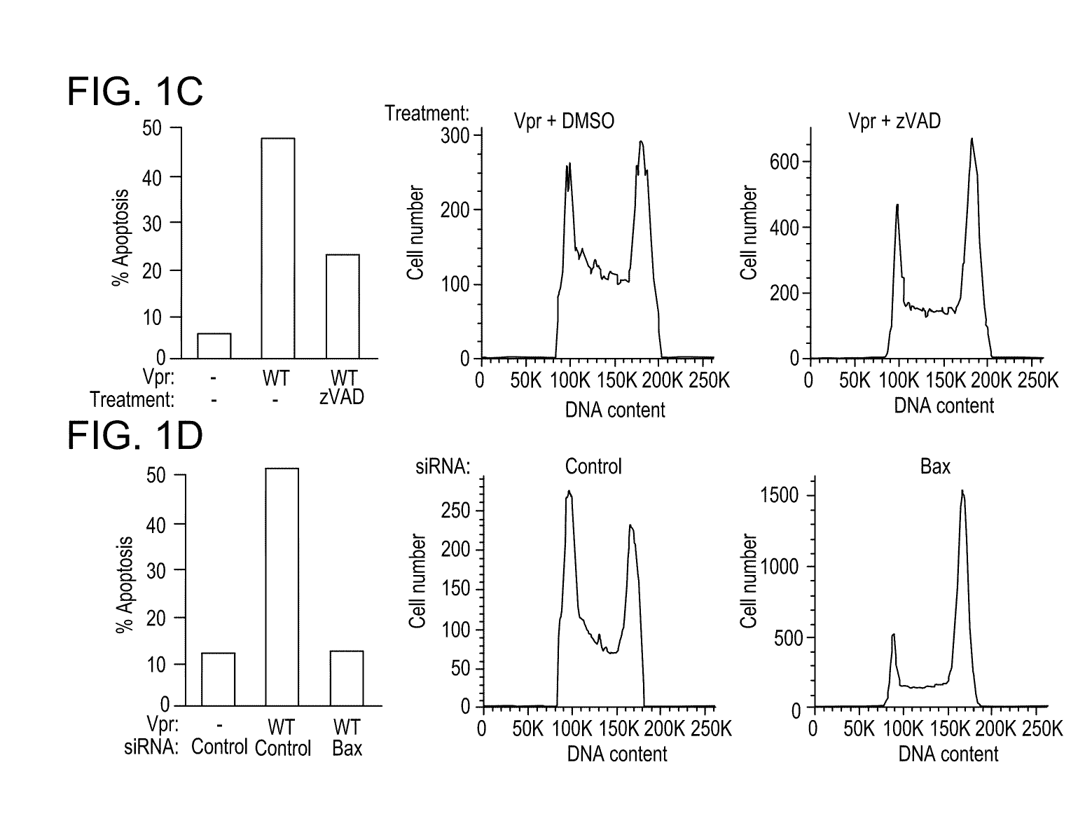

In establishing the role of VPR in CD4 cell death, applicants first examined whether it was possible to dissociate the effects of HIV-1 VPR on apoptosis and cell cycle arrest in cell culture. Initial studies were performed on the well-characterized human renal epithelial cell line, 293T. Cells were transduced with a lentivirus encoding wild type VPR or an inactive VPR mutant (Q65R) that served as a negative control¹¹. Expression of VPR remarkably increased apoptosis, determined by the percentage of cells with DNA content lower than the normal diploid amount (FIG. 1a, left panel). At the same time, VPR increased the accumulation of cells at the G2/M phase of the cell cycle (FIG. 1a, right panel). Apoptosis induced by VPR required cell cycle progression to G2/M as evidenced by the dramatic reduction in cell death observed in cells blocked in G1/S by expression of p27kip1, an inhibitor of G1 cyclin-dependent kinases (FIG. 1b). Two known inhibitors of VPR-induced cell death^{12,13} were next analyzed for their effect on cell cycle progression. The broad caspase inhibitor, zVAD-fmk, significantly reduced VPR-induced apoptosis but did not reverse the G2/M blockade (FIG. 1c, left and right panels respectively). Similarly, knockdown of Bax, a mediator of the mitochondrial death pathway, strongly suppressed VPR-induced apoptosis but did not diminish its cell cycle effect (FIG. 1d, left and right panels respectively). Together, these results demonstrated that progression into the G2/M phase of the cell cycle was necessary but not sufficient for VPR-induced apoptosis and that cell death was Bax- and caspase-dependent; however, the steps responsible for activation of this pathway were unknown.

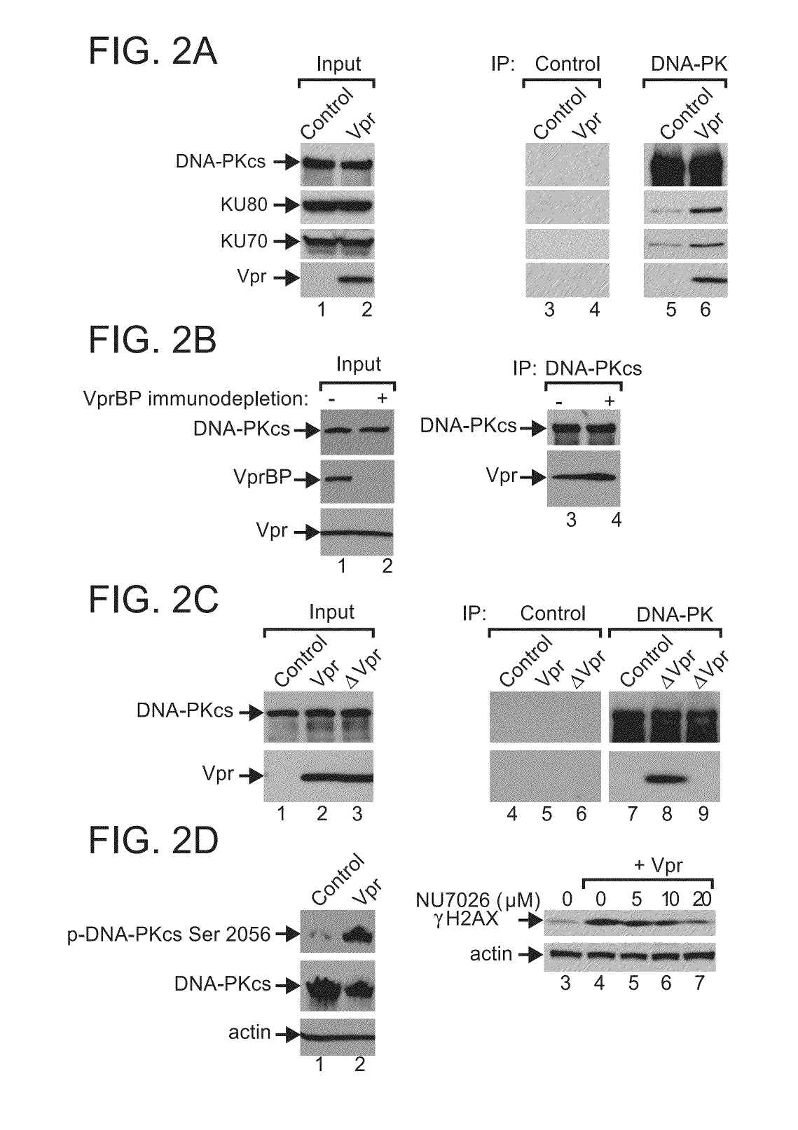

To define the mechanism that initiated cell death, we analyzed proteins associated with VprBP, which forms a complex with VPR and the Cul4-DDB1 E3 ubiquitin ligase to stimulate proteasomal degradation of selected cellular proteins¹⁰. VprBP and DNA-PK are found in a common complex in the absence of VPR¹⁴ but the potential association of DNA-PK with VPR had not been previously explored. We found that VPR specifically co-immunoprecipitated with DNA-PKcs, (FIG. 2a, lane 4 vs. 6, bottom panel) and also modestly increased its association with the with the Ku70/80 regulatory subunits (FIG. 2a, lane 5 vs. 6, middle panels). Importantly, VPR binding to DNAPK was observed after complete immunodepletion of VprBP (FIG. 2b, lane 4), demonstrating that VPR interacted with DNA-PK independently of VprBP. VPR binding to DNA-PKcs was not observed with the inactive VPR Q65R mutant (FIG. 2c, lanes 8 vs. 9), demonstrating a correlation between VPR binding to DNA-PKcs and its functional effects.

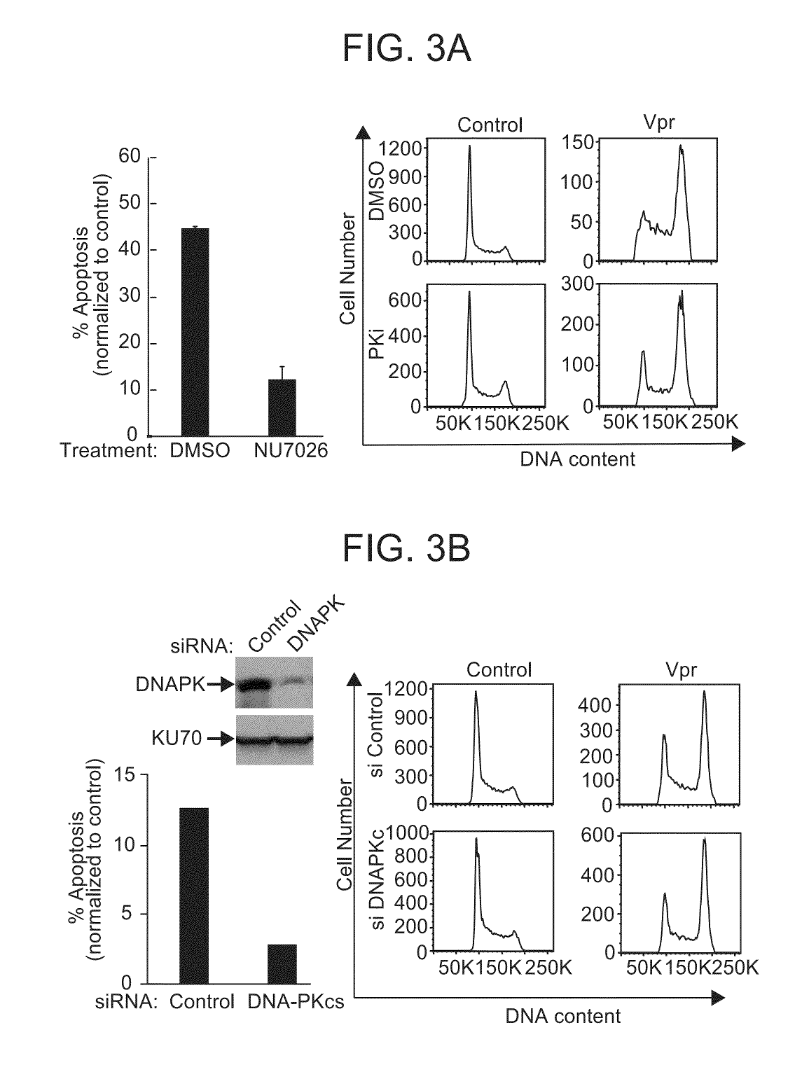

VPR stimulated the catalytic activity of DNA-PK, as evidenced by DNA-PKcs autophosphorylation (FIG. 2d, lanes 1 vs. 2, upper panel), suggesting that VPR binding to DNA-PK could activate this kinase. This activation was further examined using H2AX, a known DNA-PK substrate⁵ and a marker associated with VPR-induced damage response¹⁵. We employed NU7026, a highly specific chemical DNA-PK inhibitor. VPR induced H2AX phosphorylation that was diminished by this DNA-PK inhibitor in a dose-dependent manner (FIG. 2d, lane 4 vs. 5-7), suggesting DNA-PK catalytic kinase activity is involved in VPR-induced signaling. We next directly examined the effects of this DNA-PK inhibitor on VPR function. Strikingly, treatment with the DNA-PK inhibitor alleviated VPR-induced apoptosis in 293T cells, lowering the apoptotic activity without diminishing G2/M cell cycle arrest (FIG. 3a). To define the role of DNA-PK in VPR-induced cell death further, 293T cells were transfected either with an siRNA specifically targeting DNA-PKcs or a control, non-targeting siRNA. Transfection with the DNA-PKc siRNA strongly diminished DNA-PKc protein levels in 293T cells (FIG. 3b). Similar to DNA-PK inhibitor, knockdown of DNA-PKc significantly decreased the apoptotic effects of VPR without reducing the G2/M blockade. Collectively, these results indicate that DNA-PK is required for apoptosis induced by VPR. Thus a small interfering RNA (siRNA) against DNA-PK that specifically abolishes its expression is shown to effectively reduce cell death following HIV-1 infections. Methods of treating HIV infection/AIDS by providing a siRNA against DNA-PK are further provided herein.

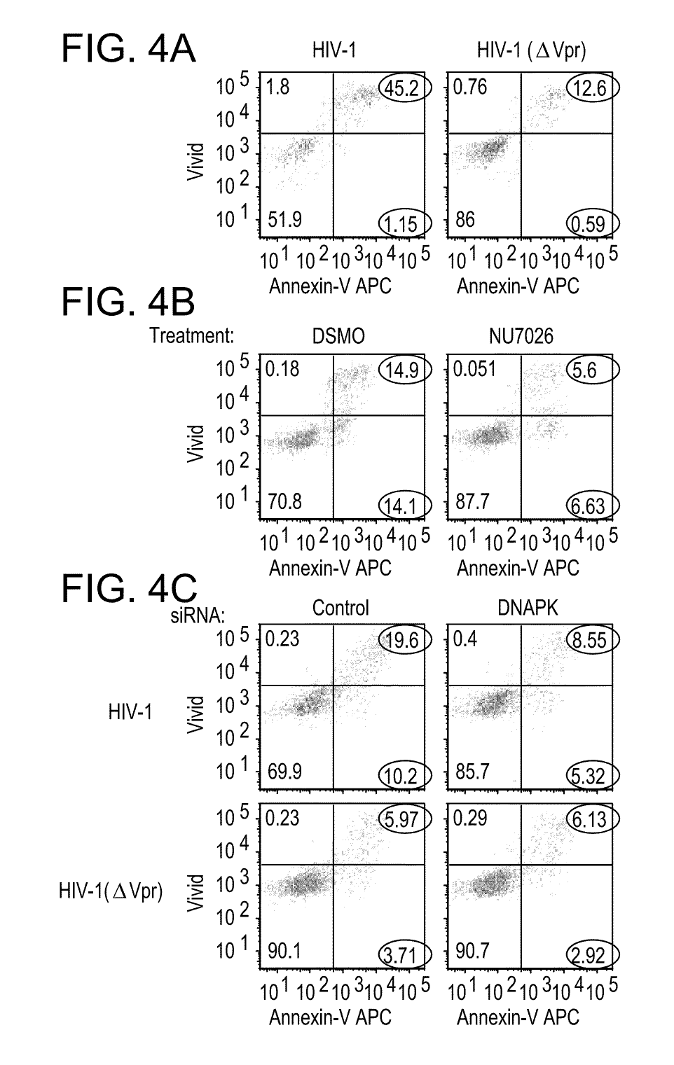

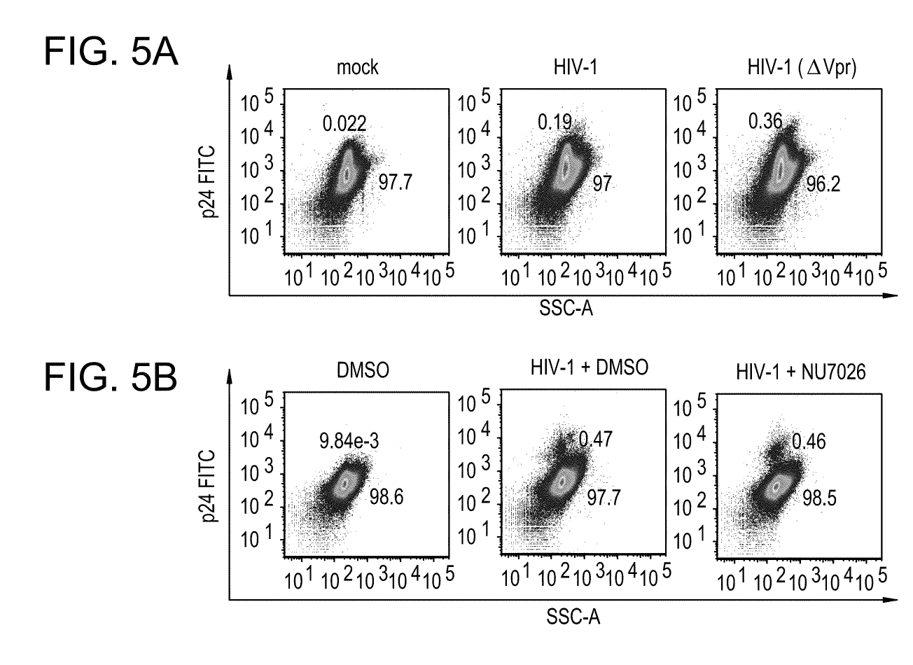

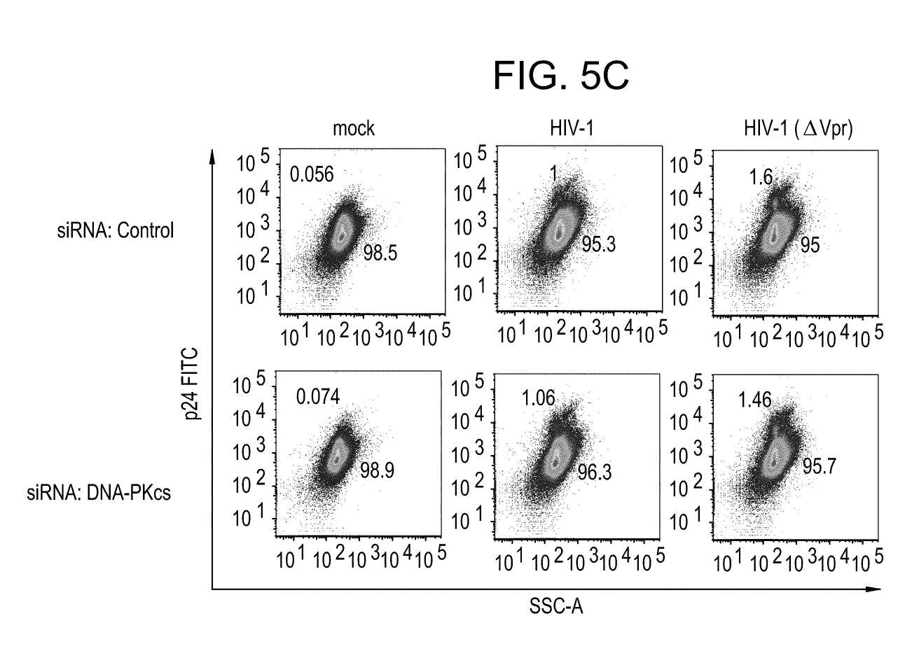

To determine whether VPR induced cell death through DNA-PK after infection with replication-competent HIV-1, we compared the effects of pathogenic HIV-1_{89.6} and a matching, Vpr-knockout (ΔVPR) virus¹⁶ in primary human CD4 cells. Infected cells were identified by p24 staining, and viability was monitored using combined staining with Annexin V and the amine reactive viability dye ViVid. Replication of both viruses was comparable as revealed by similar levels of p24 staining (FIG. 5a and data not shown). Cell death was induced in infected cells in a time-dependent manner, reaching nearly 50% death by day 12 (FIG. 4a, upper and lower right panels). In contrast, death was reduced to background levels in cells infected with ΔVPR HIV-1_{89.6} (FIG. 4a, ΔVPR). These results demonstrate that VPR mediates cell death in primary CD4 cells infected with replication-competent HIV-1.

We next asked whether DNA-PK is responsible for HIV-1 cytopathic effects in human CD4 cells. CD4⁺ lymphocytes were infected with wild type HIV-1_{89.6} and treated with either DMSO vehicle or DNA-PK inhibitor, NU7026. Because of the limited half life of this drug, cell death was measured at an earlier time point, 6 days after infection, at which levels of apoptosis were lower. Nonetheless, treatment with NU7026 profoundly diminished direct cell killing by the virus compared to the control vehicle (FIG. 4b). The frequency of infection, determined by p24 positivity using flow cytometry, remained unchanged by treatment with DNA-PK inhibitor (FIG. 5b), excluding the possibility that decreased viral infectivity accounted for the diminished death.

To document the role of DNA-PK in HIV-1 cytopathicity further, a knockdown experiment was conducted in primary human CD4⁺ T cells. Uninfected CD4 cells were transfected by nucleofection with either a non-targeting, control siRNA or a DNA-PKcs-specific siRNA. The siRNAs were mixed with a Cy3-labelled siRNA indicator, allowing positive selection of nucleofected cells on the following day. Transfected lymphocytes were then infected with either wild type HIV-1_{89.6} or ΔVPR HIV-1_{89.6} virus. While DNA-PKcs knockdown did not affect the infectivity of either virus (FIG. 5c), death of HIV-1_{89.6} infected cells was diminished by DNA-PKcs knockdown to levels comparable to ΔVPR HIV-1_{89.6} (FIG. 4c). As expected, no difference was observed in viability of DNA-PKcs and control knockdown cells after infection with ΔVPR virus. Together, these results demonstrate that a VPR-activated DNA-PK is responsible for death of primary CD4⁺ lymphocytes infected with HIV-1.

DNA-PK inhibitors inhibited bystander cell death during HIV infection. Applicants used an in vitro system in which the highly permissive cell line, CEM174 was infected by HIV-1. HIV-1 infection of the cell line causes massive killing of neighboring cells. This system is believed to mimic the situation during in vivo infection because studies in human tissues indicate that bystander death is a prominent form of death occurring during HIV-1 infection. The DNA-PK inhibitors prevented cell death despite ongoing viral replication. The effect on cell death was specific for HIV-1, as cell killing induce by DNA damaging agents was not affected by the inhibitors. Also, the pan caspase inhibitor Z-AVAD which inhibits classical apoptosis did not affect cell death by HIV-1 in this system.

Our results demonstrate that VPR plays a fundamental role in direct HIV-1 cytopathicity in primary human CD4⁺ lymphocytes through activation of the DNA-PK holoenzyme. While DNA-PK was responsible for initiating cell death, it had no affect on G2/M cell cycle arrest. At the same time, though G2/M arrest is required for cell death induction by VPR¹⁰, growth arrest alone does not promote cell death. DNA-PK is involved in G2 checkpoint maintenance, in response to DNA damage in normal cells through phosphorylation of caspase-2,7. Caspase-2 plays a role in cell death during mitotic catastrophe¹⁸, a process implicated in SIV-induced apoptosis associated with VPR¹⁹. Thus, the interaction with DNA-PK provides the critical link that activates this pathway and regulates cell viability during HIV-1 infection in T cells. HIV-1 also forms a double-stranded DNA intermediate with non-homologous ends, and DNA-PK subunits have been found in association with the viral preintegration complex²⁰, and infection could potentially further enhance DNA-PK activity through this intermediate. In proliferating HIV-1-infected cells, we suggest that VPR stimulates DNA-PK activity while causing G2/M arrest, providing a mechanism that promotes apoptosis.

It is well known that VPR is not required for productive HIV-1 replication in primary CD4⁺ cells. Yet this viral gene product confers a selective advantage to lentiviruses: it is highly conserved and retained in both primate and human lentiviruses. It has been suggested that VPR enables HIV-1 replication in non-dividing cells, particularly monocyte/macrophages, by promoting nuclear import of the viral preintegration complex^{21,22} and enhancing viral transcription²³⁻²⁶. In SIVsm, the related Vpx protein exerts a similar effect and also interacts with VprBP to permit viral infection in macrophages and in monocyte-derived dendritic cells¹⁰. Furthermore, expression of VPR in G1/S arrested cells fails to promote apoptosis (FIG. 1b). Taken together, these data suggest that the cytolytic effect of HIV-1 infection results from the action of VPR on DNA-PK in proliferating CD4⁺ T cells during the G2/M phase of the cell cycle.

Our findings are consistent with previous clinical studies of HIV-1 infected subjects, in which VPR mutations have been associated with reduced pathogenicity and slower disease progression^{27,28}. Although other viral gene products have been implicated with such outcomes in different studies²⁹, we suggest that in some cases, these effects often result from diminished viral replication that also decreases VPR gene expression in dividing primary T cells. Massive elimination of productively infected CD4⁺ T cells in various tissues during acute HIV or SIV infection sets the stage for disease progression and the development of AIDS¹⁻³. Identification of DNA-PK as the intermediary of VPR-induced cell death raises the possibility that pharmacologic intervention may help reduce the pathogenicity of infection and delay the progression of HIV-1 disease.

Our studies provide proof of principal that small molecule inhibitors of DNAPK can inhibit CD4⁺ T cell depletion, the fundamental process leading to AIDS following HIV-1 infection. Compounds that target an invariant host kinase rather than virus gene products offer the further advantage of reducing the chance of a viral escape mutation causing drug resistance.

### Chemical Description

In addition to compounds of Formula I described above the DNA-PK inhibitor can be

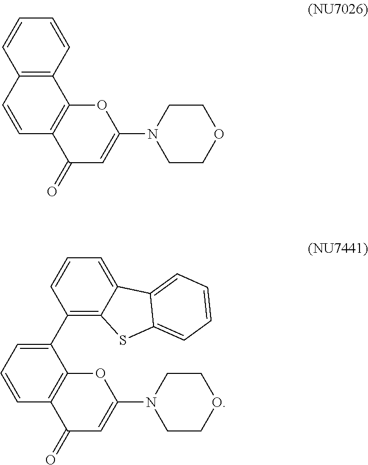 [SMILES: O=c1cc(N2CCOCC2)oc2c1ccc1ccccc12 ; O=c1cc(N2CCOCC2)oc2c(-c3cccc4c3sc3ccccc34)cccc12]

The DNA-PK inhibitor can also be a compound of Formula I in which any one or more of the following conditions is met:

(1) A₁ is N, A₂ is O, and A₃ is O.

(2) R₄ is hydrogen, halogen, C₁-C₂ alkyl, or C₁-C₂alkoxy.

(3) A₅ is CR₅, A₆ is CR₆, and A₇ is CR₇.

(4) R₅ and R₆ are independently chosen from hydrogen, halogen, C₁-C₂ alkyl, and C₁-C₂alkoxy.

(5) R₇ and R₈ are joined to form an optionally substituted phenyl ring.

(6) R₇ and R₈ are joined to form a phenyl ring that is unsubstituted or substituted with 1, 2, or 3 substituents independently chosen from hydrogen, halogen, hydroxyl, cyano, amino, thiol, C₁-C₄alkyl, C₁-C₄alkoxy, mono- or di-C₁-C₄alkylamino, C₁-C₂haloalkyl, and C₁-C₂haloalkoxy.

(7) R₇ and R₈ are joined to form an unsubstituted phenyl ring.

(8) R₈ is an optionally substituted group of the formula

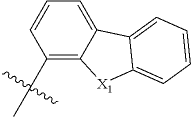

in which X₁ in O or S.

(9) R₈ is

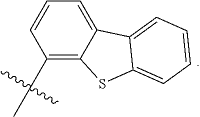 [SMILES: *c1cccc2c1sc1ccccc12]

### Methods of Treatment

Methods of treatment include providing certain dosage amounts of a DNA-PK inhibitor, such as a compound of Formula I, to a patient. The DNA-PK inhibitor may be the only compound provided to the patient or may be provided to the patient together with one or more other active agents. Dosage levels of each active agent of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the patient treated and the particular mode of administration. Dosage unit a compound of the invention. In certain embodiments 25 mg to 500 mg, or 25 mg to 200 mg of a compound of the invention are provided daily to a patient. Frequency of dosage may also vary depending on the compound used and the particular disease treated. However, for treatment of most infectious disorders, a dosage regimen of 4 times daily or less is preferred and a dosage regimen of 1 or 2 times daily is particularly preferred.

Further provided herein is a method of inhibiting CD4 cell death in a patient infected with HIV-1 comprising performing a count of CD4 cells in the patient's blood; and administering an effective amount of a DNA-PK inhibitor to the patient.

The DNA-PK inhibitor may be a compound or salt of Formula I or any of the subformula of Formula I discussed in the preceding section.

In certain embodiments the amount of DNA-PK inhibitor administered to a patient is an amount that provides an in vivo concentration of the inhibitor that is sufficient to inhibit the binding of VPR to DNA-PK in vitro.

In certain embodiments the DNA-PK inhibitor is administered once daily, once every 48 hours, twice weekly or once weekly. In certain embodiments the effective amount is an amount effective so that when a count of the CD4 cells in a patients blood is performed after 1 month, 2 months, or 6 months the number of CD4 cells either shows no statistically significant decrease, or has decreases by less that 20 percent.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease in the patient undergoing therapy.

### EXAMPLES

### General Methods

Cell cycle analysis in 293T cells was performed on ethanol-fixed cells using the propidium iodide staining method after transduction with lentiviral vectors encoding wild type or mutant VPR. Cell debris was excluded from the analysis, and the DNA content of cells below the diploid level, sub-G1, was used to calculate the apoptosis rate. The association of VPR with VprBP and DNA-PK was determined by immunoprecipitation. Briefly, whole cell extracts were prepared in lysis buffer and incubated with the relevant antibodies, concentrated on Protein G beads, and analyzed by Western blotting. HIV-1 viral stocks were generated by transfecting 293T cells with the HIV-1_{89.6} wild type and IVPR plasmids followed by propagation in CEMx174 cells as previously described¹⁶. CD4⁺ T cells were isolated from elutriated lymphocytes using magnetic bead purification, activated and infected as described in Methods. For siRNA transfections in CD4⁺ lymphocytes, isolated CD4 cells were transduced by nucleofection. In some experiments, cells were treated with either DMSO or the DNA-PK inhibitor NU7026 (Sigma) during the infection period. Viability of infected primary CD4⁺ T cells was performed using combined staining with Annexin V-APC (BD Pharmigen) and the amine reactive viability dye ViVid (Invitrogen). Flow cytometry was performed with a LSR II cell analyzer.

### Example 1

### Plasmids

The HIV-1_{89.6} and HIV-1_{89.6} ΔVPR constructs were a kind gift from Dr. Ronald Collman and were previously described^{16,30}. pHR-VPR and pHR-VPR Q65R were a kind gift from Dr. Vicente Planelles and were described previouslyl^{1,15}. The pEGFP-VPR plasmid was obtained through the NIH AIDS Research and Reference Reagent Program, Division of AIDS, NIAID, NIH: pEGFP-VPR (cat#11386) from Dr. Warner C. Greene³⁰. To generate the plasmid 1012HA-VPR the VPR open reading frame (ORF) was PCR amplified from VPR-pEGFP using the following primers:

Forward: 5′ cacttctagaatgtacccatacgatgttccagattacgctgaacaagccccagaagaccaagggccacag (SEQ ID. NO. 1) and Reverse: 5′ cactggatcctaggatctactggctccatttc. (SEQ ID. NO. 2). The PCR product was digested with XbaI and BamHI and cloned into an expression vector containing a CMV promoter digested with the same enzymes. To generate the 1012HA-VPR Q65R plasmid, mutagenesis was performed using a Stratagene Quick Change Site-Directed Mutagenesis Kit with the following primers: Forward: 5′ taataagaattctgcgacaactgctgtttat (SEQ ID. NO. 3) and reverse: 5′ ataaacagcagttgtcgcagaattcttatta. (SEQ ID. NO. 4) The PCR products were digested with DpnI at 37° C. for 1 h per the manufacturer's protocol and transformed into TOP10 bacteria (Invitrogen).

### Example 2

### Cells, Transfections and Cell Cycle Analysis

CEMX174 cells were cultured in RPMI with 10% FBS and antibiotics (penicillin and streptomycin). HEK293T cells were cultured in DMEM supplemented with 10% FBS and antibiotics (penicillin and streptomycin). For immunoprecipitation experiments HEK293T cells were transfected with 3 μg of either 1012-HAVpr or 102-HAVpr Q65R using the LipofectAMINE 2000 reagent (Life Technologies, Inc.). For cell cycle analysis HEK293T cells were harvested, washed with ice-cold PBS and resuspended in 70% cold ethanol while vortexing. The cells were incubated at −20° C. overnight, washed once with PBS and resuspended in PBS containing 25 μg/ml propidium iodide and 100 μg/ml RNaseA for 15 minutes at 25° C. Stained cells were then analyzed by flow cytometry.

### Example 3

### Immunoprecipitation and Western Blots

Cell extracts for Western blots and immunoprecipitations were made in cell lysis buffer (Cell Signal). Immunoprecipitations were performed overnight at 4° C. using normal rabbit serum, anti-VprBP or anti-DNA-PK polyclonal antibodies (Bethyl Laboratories). Protein G beads (Invitrogen) were then added for 2 h, washed three times with 1× lysis buffer, boiled and loaded onto 4-15% polyacrylamide Tris Glycine gels (BioRad). The primary antibodies used for Western blotting were anti-DNA-PK, anti-VprBP (Bethyl Laboratories), anti-actin (Sigma), anti-γH2AX (Cell Signaling), anti-HA (Santa Cruz Biotechnology), anti-Ku70, and anti-Ku80 (BD Transduction Laboratories).

### Example 4

### Lentiviral Vectors

Lentivirus vectors were produced by transiently transfecting HEK293T cells using the calcium phosphate method. Cells were transfected with pHR-VPR or pHR-VPR Q65R, together with pCMVΔR8.2ΔVPR and pHCMV-VSVG, and the media was changed the next day. Virus supernatants were collected at 48 h post transfection. The harvested supernatants were cleared by centrifugation, concentrated and frozen at −80° C. for storage.

### Example 5

### Generation of HIV-1 Stocks

The HIV-1_{89.6} and HIV-1_{89.6} ΔVPR constructs were transfected into HEK293T cells and the supernatants were collected 48 h after transfection. The viruses were then amplified for 7-10 days in CEMX174 and the supernatants were harvested, clarified by low speed centrifugation, filtered and concentrated using Centricon Plus-70. Virus stocks were frozen at −80° C. for storage.

### Example 6

### T-Cell Isolation and Nucleofection

CD4⁺ T lymphocytes were isolated from elutriated lymphocytes prepared from blood of healthy donors by negative selection with a CD4⁺ T-cell isolation kit II (Miltenyi Biotech) according to the manufacturer's instructions. The purity of the isolated CD4⁺ T cells was assessed by fluorescence-activated cell sorting (FACS) analysis for CD4 (BD-Pharmingen) and 95% of the cells were CD4⁺ upon isolation. The cells were stimulated with phytohaemagglutinin (0.5 μml⁻¹) (Remel) and 20 U ml⁻¹ IL-2 (Peprotech) for 24 h and maintained in 20 U ml⁻¹ IL-2. For nucleofection, activated CD4⁺ T cells (0.5×10⁷) were resuspended in 100 μl of T-cell Nucleofector reagent and immediately electroporated with the recommended protocol T-020 using the Nucleofector instrument (Amaxa Biosystems). The cells were nucleofected with Cy3-labeled siGLO indicator together with either non-targeting, control siRNA or DNA-PK siRNA (Dharmacon Co.). For each transfection, a mix containing 800 nM unlabelled siRNA and 400 nM Cy3-labelled siRNA was used. The cells were washed and sorted 12 h after nucleofection using FACSARIA Cell Sorter II (BD), and maintained in RPMI containing 20 U ml⁻¹ IL-2 (PeproTech).

### Example 7

### HIV-1 Infection

Purified, activated CD4⁺ T cells were infected 2-3 days after isolation. For nucleofected cells, infection was done 36 h after nucleofection. HIV-1 infection of CD4⁺ T lymphocytes was performed in 96-well round-bottomed culture plates by combining 10 μl virus stock with 150 μl CD4⁺ T lymphocytes (1.5×10⁵ cells). The multiplicity of infection was ˜1.0. CD4⁺ T lymphocytes were harvested at the indicated times following exposure to virus.

### Example 8

### Flow Cytometric Analysis of HIV-1 Infected Cells

CD4⁺ T lymphocytes were harvested for Annexin V, Vivid and intracellular p24-Gag staining at the indicated times following exposure to virus. In brief, the cells were washed and stained with Annexin V-APC (BD Pharmigen) and Vivid (Invitrogen) for 20 min. Cells were washed once, fixed, and permeabilized with Cytoperm/Cytofix (BD Pharmingen) for 20 min. Cells were then stained for p24 Gag (KC-57 fluorescein isothiocyanate (FITC); Coulter) for 30 min and washed once in 1× Perm/wash buffer (BD Pharmingen). Flow cytometry was performed with an LSR II cell analyzer (Becton Dickinson), and data analysis was performed with FlowJo software (Tree Star, Inc.)

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

### REFERENCES

1. Ho, D. D. et al. Rapid turnover of plasma virions and CD4 lymphocytes in HIV-1 infection. Nature 373, 123-126 (1995).
2. Mattapallil, J. J. et al. Massive infection and loss of memory CD4+ T cells in multiple tissues during acute SIV infection. Nature 434, 1093-1097 (2005).
3. Nishimura, Y. et al. Resting naive CD4+ T cells are massively infected and eliminated by X4-tropic simian-human immunodeficiency viruses in macaques. Proc Nall Acad Sci USA 102, 8000-8005 (2005).
4. Smith, G. C. & Jackson, S. P. The DNA-dependent protein kinase. Genes Dev 13, 916-934 (1999).
5. Mukherjee, B. et al. DNA-PK phosphorylates histone H2AX during apoptotic DNA fragmentation in mammalian cells. DNA Repair (Amst) 5, 575-590 (2006).
6. McCune, J. M. The dynamics of CD4+ T-cell depletion in HIV disease. Nature 410, 974-979 (2001).
7. Hazenberg, M. D., Hamann, D., Schuitemaker, H., & Miedema, F. T cell depletion in HIV-1 infection: how CD4+ T cells go out of stock. Nat Immunol 1, 285-289 (2000).
8. Gandhi, R. T. et al. HIV-1 directly kills CD4+ T cells by a Fas-independent mechanism. J Exp Med 187, 1113-1122 (1998).
9. Speirs, C. et al. Analysis of human immunodeficiency virus cytopathicity by using a new method for quantitating viral dynamics in cell culture. J Virol 79, 4025-4032 (2005).
10. Malim, M. H. & Emerman, M. HIV-1 accessory proteins—ensuring viral survival in a hostile environment. Cell Host Microbe 3, 388-398 (2008).
11. DeHart, J. L. et al. HIV-1 VPR activates the G2 checkpoint through manipulation of the ubiquitin proteasome system. Virol J 4, 57 (2007).
12. Stewart, S. A., Poon, B., Song, J. Y., & Chen, I. S. Human immunodeficiency virus type 1 vpr induces apoptosis through caspase activation. J Virol 74, 3105-3111 (2000).
13. Andersen, J. L. et al. HIV-1 VPR-induced apoptosis is cell cycle dependent and requires Bax but not ANT. PLoS Pathog 2, e127 (2006).
14. Koch, C. A. et al. Xrcc4 physically links DNA end processing by polynucleotide kinase to DNA ligation by DNA ligase IV. EMBO J. 23, 3874-3885 (2004).
15. Zimmerman, E. S. et al. Human immunodeficiency virus type 1 VPR-mediated G2 arrest requires Rad17 and Hus1 and induces nuclear BRCA1 and gamma-H2AX focus formation. Mol Cell Biol 24, 9286-9294 (2004).
16. Balliet, J. W. et al. Distinct effects in primary macrophages and lymphocytes of the human immunodeficiency virus type 1 accessory genes vpr, vpu, and nef: mutational analysis of a primary HIV-1 isolate. Virology 200, 623-631 (1994).
17. Shi, M. et al. DNA-PKcs-PIDDosome: a nuclear caspase-2-activating complex with role in G2/M checkpoint maintenance. Cell 136, 508-520 (2009).
18. Andersen, J. L. et al. Restraint of apoptosis during mitosis through interdomain phosphorylation of caspase-2. EMBO J 28, 3216-3227 (2009).
19. Paiardini, M. et al. Perturbations of cell cycle control in T cells contribute to the different outcomes of simian immunodeficiency virus infection in rhesus macaques and sooty mangabeys. J Virol 80, 634-642 (2006).
20. Li, L. et al. Role of the non-homologous DNA end joining pathway in the early steps of retroviral infection. EMBO J. 20, 3272-3281 (2001).
21. Heinzinger, N. K. et al. The VPR protein of human immunodeficiency virus type 1 influences nuclear localization of viral nucleic acids in nondividing host cells. Proc Natl Acad Sci USA 91, 7311-7315 (1994).
22. Vodicka, M. A., Koepp, D. M., Silver, P. A., & Emerman, M. HIV-1 VPR interacts with the nuclear transport pathway to promote macrophage infection. Genes Dev 12, 175-185 (1998).
23. Connor, R. I., Chen, B. K., Choe, S., & Landau, N. R. VPR is required for efficient replication of human immunodeficiency virus type-1 in mononuclear phagocytes. Virology 206, 935-944 (1995).
24. Felzien, L. K. et al. HIV transcriptional activation by the accessory protein, VPR, is mediated by the p300 co-activator. Proc Nall Acad Sci USA 95, 5281-5286 (1998).
25. Goh, W. C. et al. HIV-1 VPR increases viral expression by manipulation of the cell cycle: a mechanism for selection of VPR in vivo. Nat Med 4, 65-71 (1998).
26. Subbramanian, R. A. et al. Human immunodeficiency virus type 1 VPR is a positive regulator of viral transcription and infectivity in primary human macrophages. J Exp Med 187, 1103-1111 (1998).
27. Somasundaran, M. et al. Evidence for a cytopathogenicity determinant in HIV-1 VPR. Proc Nall Acad Sci USA 99, 9503-9508 (2002).
28. Lum, J. J. et al. VPR R77Q is associated with long-term nonprogressive HIV infection and impaired induction of apoptosis. J Clin Invest 111, 1547-1554 (2003).
29. Shedlock, D. J. et al. HIV-1 viral genes and mitochondrial apoptosis. Apoptosis 13, 1088-1099 (2008).
30. Schaeffer, E., Geleziunas, R., & Greene, W. C. Human immunodeficiency virus type 1 Nef functions at the level of virus entry by enhancing cytoplasmic delivery of virions. J Virol 75, 2993-3000 (2001).

1. A method of treating HIV-1 infection in a patient infected with an HIV-1 virus comprising providing a therapeutically effective amount of a DNA-PK inhibitor to the patient.

2. The method of claim 1 wherein The DNA-PK inhibitor is compound of the formula 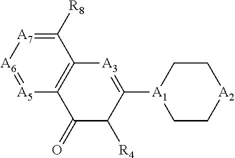
or a pharmaceutically acceptable salt thereof, wherein
A₁ is N or CH;
A₂ is NH, O, S, or CH;
A₃ is NH, O, S, or CH;
R₄ is hydrogen, halogen, hydroxyl, cyano, amino, thiol, phenyl, C₁-C₄alkyl, C₁-C₄alkoxy, mono- or di-C₁-C₄alkylamino, C₁-C₂haloalkyl, or C₁-C₂haloalkoxy;
A₅ is N or CR₅;
A₆ is N or CR₆;
A₇ is N or CR₇;
wherein not more than 2 of A₅, A₆, and A₇ are N;
R₅ and R₆ are independently chosen at each occurrence from hydrogen, halogen, hydroxyl, cyano, amino, thiol, C₁-C₄alkyl, C₁-C₄alkoxy, mono- or di-C₁-C₄alkylamino, C₁-C₂haloalkyl, C₁-C₂haloalkoxy, C₃-C₇cyclolkyl, heterocycloalkyl having 5 to 7 ring atoms with 1 or 2 ring atoms being, N, S, or O and remaining ring members being carbon;
R₇ carries the same definition as R₅ and R₆ or may be joined with R₈ to form a ring; and
R₈ is an optionally substituted heterocyclic or carbocyclic ring system having one ring or two or three fused rings each ring containing from 0 to 3 heteroatoms independently chosen from N, O, and S; or
R₇ and R₈ are joined to form an optionally substituted phenyl or pyridyl ring.

3. The method of claim 2, wherein the DNA-PK inhibitor is a compound, or salt thereof, of the formula

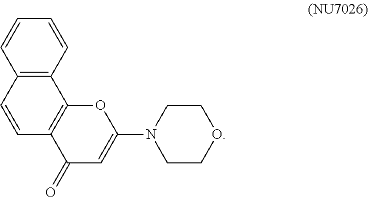 [SMILES: O=c1cc(N2CCOCC2)oc2c1ccc1ccccc12]

4. The method of claim 2, wherein the DNA-PK inhibitor is a compound, or salt thereof, of the formula

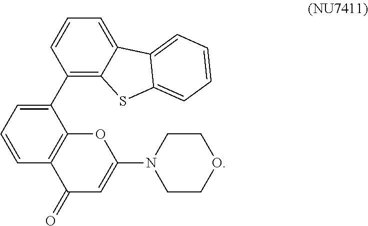 [SMILES: O=c1cc(N2CCOCC2)oc2c(-c3cccc4c3sc3ccccc34)cccc12]

5. The method of claim 2, wherein A₁ is N, A₂ is O, and A₃ is O.

6. The method of claim 2, wherein R₄ is hydrogen, halogen, C₁-C₂ alkyl, or C₁-C₂alkoxy.

7. The method of claim 2 wherein A₅ is CR₅, A₆ is CR₆, and A₇ is CR₇.

8. The method of claim 7, wherein R₅ and R₆ are independently chosen from hydrogen, halogen, C₁-C₂ alkyl, and C₁-C₂alkoxy.

9. The method of claim 5, wherein R₇ and R₈ are joined to form an optionally substituted phenyl ring.

10. The method of claim 9, wherein R₇ and R₈ are joined to form a phenyl ring that is unsubstituted or substituted with 1, 2, or 3 substituents independently chosen from hydrogen, halogen, hydroxyl, cyano, amino, thiol, C₁-C₄alkyl, C₁-C₄alkoxy, mono- or di-C₁-C₄alkylamino, C₁-C₂haloalkyl, and C₁-C₂haloalkoxy.

11. The method of claim 9, wherein R₇ and R₈ are joined to form an unsubstituted phenyl ring.

12. The method of claim 5, wherein R₈ is an optionally substituted group of the formula 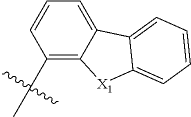
in which X₁ in O or S.

13. The method of claim 12, wherein X₁ is S.

14. The method of claim 12, wherein R₈ is

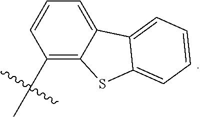 [SMILES: *c1cccc2c1sc1ccccc12]

15. The method of claim 1, wherein the DNA-PK inhibitor is provided together with instructions for treating an HIV-1 infection.

16. A method of inhibiting CD4 cell death in a patient infected with HIV-1 comprising
Performing a count of CD4 cells in the patient's blood; and
Administering an effective amount of a DNA-PK inhibitor to the patient.

17. The method of claim 16, wherein the DNA-PK inhibitor is a compound of the formula: 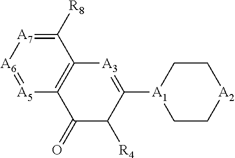
or a pharmaceutically acceptable salt thereof, wherein
A₁ is N or CH;
A₂ is NH, O, S, or CH;
A₃ is NH, O, S, or CH;
R₄ is hydrogen, halogen, hydroxyl, cyano, amino, thiol, phenyl, C₁-C₄alkyl, C₁-C₄alkoxy, mono- or di-C₁-C₄alkylamino, C₁-C₂haloalkyl, or C₁-C₂haloalkoxy;
A₅ is N or CR₅;
A₆ is N or CR₆;
A₇ is N or CR₇;
wherein not more than 2 of A₅, A₆, and A₇ are N;
R₅ and R₆ are independently chosen at each occurrence from hydrogen, halogen, hydroxyl, cyano, amino, thiol, C₁-C₄alkyl, C₁-C₄alkoxy, mono- or di-C₁-C₄alkylamino, C₁-C₂haloalkyl, C₁-C₂haloalkoxy, C₃-C₇cyclolkyl, heterocycloalkyl having 5 to 7 ring atoms with 1 or 2 ring atoms being, N, S, or O and remaining ring members being carbon;
R₇ carries the same definition as R₅ and R₆ or may be joined with R₈ to form a ring; and
R₈ is an optionally substituted heterocyclic or carbocyclic ring system having one ring or two or three fused rings each ring containing from 0 to 3 heteroatoms independently chosen from N, O, and S; or
R₇ and R₈ are joined to form an optionally substituted phenyl or pyridyl ring.
